(19) United States
(12) Patent Application Publication
(10) Pub. No.: US 2022/0392629 A1
(43) Pub. Date: Dec. 8, 2022

(54) PORTABLE HEALTHCARE SYSTEM INSTALLED AT A REMOTE LOCATION HAVING LIMITED INTERNET CONNECTIVITY

(71) Applicant: Sandeep REDDY, Geelong (AU)

(72) Inventor: Sandeep REDDY, Geelong (AU)

(21) Appl. No.: 17/694,819

(22) Filed: Mar. 15, 2022

Related U.S. Application Data
(60) Provisional application No. 63/196,222, filed on Jun. 2, 2021.

(51) Int. Cl. G16H 40/67; G16H 10/60; G16H 15/00; G16H 20/13; A61B 5/00; G16H 50/20; G06V 40/16

(52) U.S. Cl. CPC G16H 40/67; G16H 10/60; G16H 15/00; G16H 20/13; A61B 5/0004; A61B 5/0022; G16H 50/20; G06V 40/16

(57) ABSTRACT

A portable healthcare system installed at a remote location having limited internet connectivity, the system comprising the following features: a camera configured to receive images/video of a patient, a point-of-care testing having medical devices configured to: receive samples from a body of the patient, determine parameters based on information from the samples, a pills dispenser, configured to dispense pills, a memory unit configured to store machine-readable instructions, and a processing unit operably connected with the memory unit, the processing unit obtaining the machine-readable instructions from the memory unit, and being configured by the machine-readable instructions to: run facial recognition based on the captured image of the patient and identify the patient, compare the determined parameters with predefined parameters, determine a health related information of the human body based on the comparison, and determine a medical condition of the patient, based on the health related information, connect a health professional, located at a distant location, through a augmented reality (AR) lens with the patient, record conversation between the patient and the health professional, and instruct the pills dispenser to dispense the pills, a communication means configured to establish a communication network which is configured to send the health related information, and the medical condition to user devices held by the patient, and/or health organizations, a user interface, configured to display the virtual health professional and facilitate interactive communication between the virtual health professional and the patient.

### TECHNICAL FIELD

The present invention relates in general to healthcare technologies and specifically, to a portable healthcare system installed at a remote location having limited internet connectivity.

### BACKGROUND

Despite the huge infrastructural and healthcare differences between developing and developed countries, access is a common issue in rural health areas around the world. Even in the countries where most of the population lives in rural areas, the resources are concentrated in the cities. All countries have difficulties with transport and communication, and they all face the challenge of shortages of doctors and other healthcare professionals in rural and remote areas. Many rural people are caught in the poverty-ill health-low productivity downward spiral, particularly in developing countries.

Rural and remote areas alone encompasses many diverse locations and communities and people living in these areas face unique challenges due to their geographic isolation. Those living outside metropolitan areas often have poorer health outcomes compared with those living in metropolitan areas. For example, data shows that people living in rural and remote areas have higher rates of hospitalizations, mortality, injury and poorer access to, and use of, primary health care services, compared with those living in metropolitan areas.

Health inequalities in rural and remote areas may be due to factors, including:
challenges in accessing health care or health professionals, such as specialists,
social determinants such as income, education, and employment opportunities,
higher rates of risky behaviors such as tobacco smoking and alcohol use, and
higher rates of occupational and physical risk, for example from farming or mining work and transport-related accidents.

Around the world, the health status of people in rural areas is generally worse than in urban areas. In South Africa, infant mortality rates in rural areas are 1.6 times that of urban areas. Rural children are 77% more likely to be underweight or under height for age; 56% of rural South Africans live 5 km from a health facility: and 75% of South Africa's poor people live in rural areas.

On average, citizens living in rural and remote areas have shorter lives, higher levels of disease and injury and poorer access to and use of health services, compared with people living in metropolitan areas. Poorer health outcomes in rural and remote areas may be due to multiple factors including lifestyle differences and a level of disadvantage related to education and employment opportunities, as well as access to health services.

Rural and remote parts of the world lack or have limited access to appropriate primary and acute care healthcare services. Traditional facilities are dependent on human healthcare professionals to deliver healthcare. The dependency leads to not only limited services but also in many cases safety and quality issues. Also, lack of timely access to healthcare can lead to deterioration of medical conditions and increased healthcare costs.

The situation has led to gross health inequalities with health status of people in rural areas generally worse than their urban counterparts. This means people living in rural and remote areas have shorter lives and higher levels of disease.

Recently, the WHO has initiated the ‘Towards Unity for Health’ (TUFH) project. The project intends to study and promote efforts worldwide to create unity in health service organizations—particularly through a sustainable integration of medicine and public health, or, in other words, of individual health and community health related activities—and consider the implication for important reforms within the health professions, practice and education. It is in rural practice that integration of health care is well exemplified. As well as knowing the health care needs of individual patients, the rural doctor must understand the needs of the community and its resources for health. Many of the issues raised in this working paper are particularly pertinent in rural and remote areas. Innovative models of health service delivery have been developed by rural practitioners. There is potential for the use of communication information technology and tele-health in serving the needs of rural people.

Current healthcare models like fly in fly out services or telemedicine services to address the aforementioned issues are not adequate or not sustainable on the long term. These models are either expensive to maintain or have infrastructural and logistical limitations, which don't allow for early intervention or continuity of care.

There is a requirement to consider locally situated and long-term solutions to address limited healthcare access in rural and remote areas. Current and future technology, particularly biotechnology and information technology, presents an opportunity to offer healthcare in these areas with minimal or no involvement of human healthcare professionals thus limiting the dependency on human healthcare professionals.

Considering the above-mentioned problems, it is highly desirable to have a portable healthcare system installed at a remote location having limited internet connectivity that can be deployed at any remote location and can provide healthcare services over the satellite communication.

### SUMMARY

Embodiments of the present disclosure present technological improvements as solutions to one or more of the above-mentioned technical problems recognized by the inventor in conventional solutions.

According to an aspect of the present invention there is provision of a portable healthcare system installed at a remote location having limited internet connectivity, the system comprises a camera configured to receive images/video of a patient, a point-of-care testing having one or more medical devices wherein any medical device of the one or more medical devices configured to receive one or more samples from a body of the patient, determine one or more parameter based on information from the one or more samples, a pills dispenser, configured to dispense one or more pills, a memory unit configured to store machine-readable instructions, and a processor operably connected with the memory unit, the processor obtaining the machine-readable instructions from the memory unit, and being configured by the machine-readable instructions to run facial recognition based on the captured image of the patient and identify the patient, compare the determined one or more parameters with predefined parameters, determine a health related information of the human body based on the comparison, and determine a medical condition of the patient, based on the health related information, connect a health professional, located at a distant location, through an augmented reality (AR) lens with the patient, record conversation between the patient and the health professional using natural language processing, and instruct the pills dispenser to dispense the one or more pills, a communication means configured to establish a communication network which is configured to send the health related information, and the medical condition to one or more user devices held by the patient, and/or health organizations, a user interface, configured to display the virtual health professional and facilitate interactive communication between the virtual health professional and the patient to convey the health related information and the medical condition to the patient, a solar power generator configured to provide power, a printer configured to print the recorded conversation, the health related information and the medical condition. Further, the augmented reality (AR) lens configured to project the health professional image as a hologram. In addition, conversation recorded between the patient and the health professional using natural language processing is used to create an automatic medical advisory for other patients having same medical issues. Furthermore, the communication means configured to establish communication network by using satellite communication to ensure uninterrupted communication between the patient and the health professional

In accordance with an embodiment of the present invention, the one or more medical devices are selected from a group comprising oxygen meter, electronic blood pressure (BP) monitor, thermometer, uric acid/UREA profiler, urine analyzer, glucose analyzer and cardiovascular analyzer, ultrasound scanner, an ECG analyser or a combination thereof.

In accordance with an embodiment of the present invention, one or more parameters are selected from a group comprising body temperature, heart rate, SpO₂, platelet count, salivary flow, pH and buffering capacity of saliva, Glucose, Bilirubin, Ketone, Specific Gravity, Blood, pH, Protein, Urobilinogen, Nitrite and Leukocyte from urine for testing of hematology, systolic blood pressure, blood gas, pathogens of certain infectious diseases, pregnancy, cardiac markers, and glucose.

In accordance with an embodiment of the present invention, the processor is further configured to determine emotions, and mental state of the patient based on the captured images/video using facial recognition.

Additional aspects, advantages, features and objects of the present disclosure would be made apparent from the drawings and the detailed description of the illustrative embodiments construed in conjunction with the complete specification that will follow.

It will be appreciated that features of the present disclosure are susceptible to being combined in various combinations without departing from the scope of the present disclosure.

### BRIEF DESCRIPTION OF DRAWINGS

So that the way the above recited features of the present invention can be understood more clearly, embodiments are illustrated in the appended drawings. It is to be noted, however, that the appended drawings illustrate only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein:

FIG. 1 illustrates a portable healthcare system installed at a remote location having limited internet connectivity, in accordance with an embodiment of the present invention;

FIG. 2, FIG. 3, and FIG. 4 illustrate information flow using system of FIG. 1, in accordance with an embodiment of the present invention; and

FIG. 5 illustrate an installation implementing the system of FIG. 1, in accordance with an embodiment of the present invention.

### DESCRIPTION OF EMBODIMENTS

The present invention is described hereinafter by various embodiments with reference to the accompanying drawing, wherein reference numerals used in the accompanying drawing correspond to the like elements throughout the description.

While the present invention is described herein by way of example using embodiments and illustrative drawings, those skilled in the art will recognize that the invention is not limited to the embodiments of drawing or drawings described and are not intended to represent the scale of the various components. Further, some components that may form a part of the invention may not be illustrated in certain figures, for ease of illustration, and such omissions do not limit the embodiments outlined in any way. The drawings and detailed description thereto are not intended to limit the invention to the form disclosed, but on the contrary, the invention is to cover all modifications, equivalents, and alternatives falling within the scope of the present invention as defined by the appended claim. As used throughout this description, the word “may” be used in a permissive sense (i.e., meaning having the potential to), rather than the mandatory sense, (i.e., meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers, or steps. Likewise, the term “comprising” is considered synonymous with the terms “including” or “containing” for applicable legal purposes.

Further, the various embodiments described herein below include specific method steps in an exemplary order, but a wide variety of other such method steps could be implemented within the scope of the invention, including additional steps, omission of some steps, or performing the method in a different order.

The present invention aims to overcomes the drawbacks of the prior art by providing a portable healthcare system installed at a remote location having limited internet connectivity. The portable healthcare system may be made of removable walls, temporary roof, antennas, provision for electricity, to provide patients at remote locations such as villages, forests, during wars, with automated facility to get diagnosis and treatment remotely with/without supervision of a health practitioner.

The present invention is now described in detail with reference to accompanying drawings.

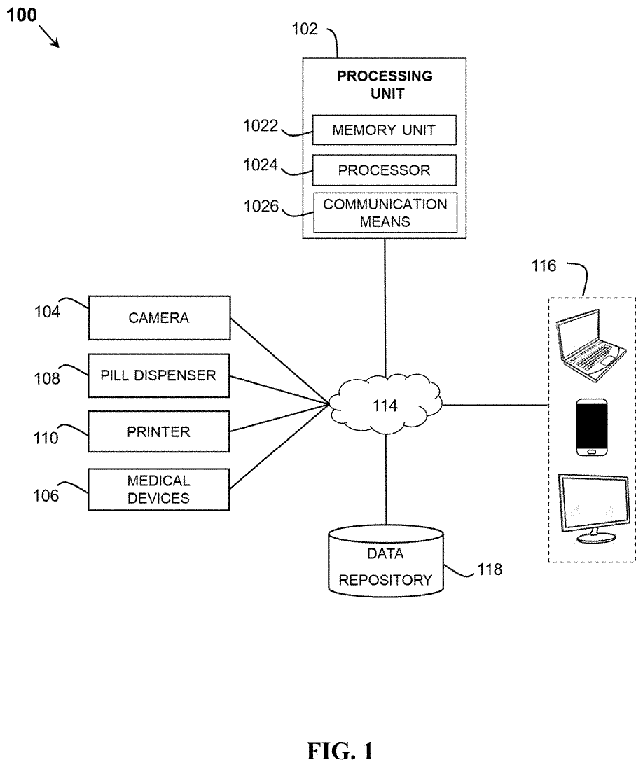

FIG. 1 depicts a portable healthcare system 100 installed at a remote location having limited internet connectivity, in accordance with an embodiment of the present invention. In an embodiment of the present invention, the system 100 comprises a camera 104, a point-of-care testing, a pills dispenser 108, a processing unit 102, a memory unit 1022, a communication means 1026, a user interface 116, a solar power generator and a printer 110.

In accordance with an embodiment of the present invention, the memory unit 1022 is configured to store machine readable instructions. The machine-readable instructions may be loaded into the memory unit 1022 from a non-transitory machine-readable medium, such as, but not limited to, CD-ROMs, DVD-ROMs and Flash Drives. Alternately, the machine-readable instructions may be loaded in a form of a computer software program into the memory unit 1022. The memory unit 1022 in that manner may be selected from a group comprising, but not limited to, EPROM, EEPROM and Flash memory unit 1022.

Furthermore, the processing unit 102 is operably connected with the memory unit 1022. In various embodiments, the processing unit 102 is one of, but not limited to, microprocessor 1024, a general-purpose processor 1024, an application specific integrated circuit (ASIC) and a field-programmable gate array (FPGA). The processing unit 102 is capable of being used for local processing and/or cloud-based remote processing. Moreover, the processing unit 102 may implement Artificial Intelligence (AI), Machine Learning (ML) and Deep Learning (DL) based technologies for, but not limited to, data analysis, collating data & presentation of data in real-time.

In accordance with an embodiment of the present invention, the camera 104 is configured to receive images/video of a patient. The captured image or the recorded video may be in color format, preferably RGB format. The camera 104 is further operable to store the captured image or video in one or more readable format on the memory unit 1022 and/or the client device 104. As per the preferred embodiment of the present invention, the one or more readable format is one of .PNG, .JPEG, or .TIFF format. The captured image/video is sent to the processing unit 102 through a communication means 1026.

The communication means 1026 is in communication with the processing unit 102 and can be a short-range communication network 114 and/or a long-range communication network 114, wired or wireless communication network 114. The communication network 114 may be implemented using a number of protocols, such as but not limited to, TCP/IP, 3GPP, 3GPP2, LTE, IEEE 802.x etc. The communication means 1026 may be wireless communication network 114 selected from, but not limited to, Bluetooth, radio frequency, internet or satellite communication network 114 providing maximum coverage. Preferably, the network 114 is internet. In a preferred embodiment, the satellite communication may be used. The satellite may be a geostationary satellite configured to relay and amplify radio telecommunication signals via a transponder. The satellite may create a communication channel between a source transmitter (the portable installation) and a receiver at different locations on Earth such as hospital, medical store, or a health care professional.

Further, the point-of-care testing may have one or more medical devices 106. The one or more medical devices 106 may be selected from a group comprising oxygen meter, electronic blood pressure (BP) monitor, thermometer, uric acid/UREA profiler, urine analyzer, glucose analyzer and cardiovascular analyzer, ultrasound scanner, an ECG analyzer or a combination thereof. The one or more medical devices 106 may be configured to provide statistics of vital activities on a body of a patient. The medical devices 106 may have, but not limited to, sensors, needles, robotic arms, pressure sensors, laser sensors, ultrasonic sensors, chemical sensors, temperature sensors, etc. to detect vital activities of the body of the patient. The one or more medical devices may send one or more parameters related to the vital activities of patient's body to the processing unit 102 over the communication means 1026.

In accordance with an embodiment of the present invention, the processing unit 102 may be in communication with the pills dispenser 108. The pills dispenser 108 may have one or more containers to store medical pills such as, but not limited to, capsules, tablets, powder etc. The one or more containers may have automated motorized dispensing mechanism connected with the processing unit 102. The automated motorized dispensing mechanism may include, but not limited to, lids, one or more electric motors, conveyer belts, sensors, electric circuits to operate lids and electric motors.

In accordance with an embodiment of the present invention, the processing unit 102 may be connected with the printer 110. The printer 110 may be selected from a group comprising laser printer, solid ink printer, LED printer, inkjet printer, multifunction printer, dot matrix printer, and 3D printer.

Further, in accordance with an embodiment of the present invention, the user interface 116 may include a display envisaged to show the data received from the medical devices 106, the processing unit 102, the one or more camera 104 and results of data analysis. The display may be, but not limited to, Light-emitting diode display (LED), electroluminescent display (ELD), liquid crystal display (LCD), Organic light-emitting diode (OLED) & AMOLED display. Furthermore, the user interface 116 may include accessories like keyboard, mouse etc. envisaged to provide input capability to enable a user to enter his/her details and to change or alter any entities. In another embodiment, the user interface 116 may be a touch input-based display that integrates the Input-output functionalities.

In accordance with an embodiment of the present invention, one or more user devices 1162 may be connected with the processing unit 102 via a wired or wireless connection. Herein, the one or more user devices 1162 may be selected from computing devices such as desktop PC, laptop, PDA or hand-held computing device such as smartphones and tablets. The one or more user devices 1162 may be associated with the patients.

In accordance with an additional or alternative embodiment of the present invention, the processing unit 102 may also include a registration unit adapted to receive one or more user details and register the one or more users on the system 100. The details may include, but not limited to, username, contact number, email ID, age, gender, etc. Besides, the registration unit may also have a biometrics receiver configured to register the one or more users via his/her biometrics. The biometrics may include, fingerprint recognition, face recognition, iris recognition, or a combination thereof. In one embodiment, the processing unit 102 may be a stand-alone device where a patient of the one or more patients may come and register himself/herself for their checkups.

In accordance with an embodiment of the present invention, medical records and data collected from the medical devices 106 and the camera 104 may be stored in a local storage (such as SSD, eMMC, Flash, SD card, etc) or a cloud-based storage. In any manner, the local storage 108 is envisaged to be capable of providing the data to the processing unit 102, when the data is queried appropriately using applicable security and other data transfer protocols.

In accordance with an embodiment of the present invention, the system 100 may be powered by a solar power generator. The solar power generation such as, but not limited to solar panels, solar trough collector. A person skilled in the art shall appreciate that instead of the solar power generator, other means of power generation may be used such as diesel set, electricity from a power grid, wind power, etc.

In accordance with an embodiment of the present invention, the system 100 may be an embedded system (where all the components of the system 100 are provided as an integral unit) or a distributed system (wherein some of the components are provided separately, for example, processing unit 102 may be a cloud processing unit 102 provided on a remote placed server and the local storage may also be a cloud-based storage).

In accordance with an embodiment of the present invention, the system 100 may also include a data repository 118. The data repository 118 may be a local storage such as SSD, eMMC, Flash, SD card, etc. or a cloud-based storage. In any manner, the data repository 118 is envisaged to be capable of providing the data to the processing unit 102, when the data is queried appropriately using applicable security and other data transfer protocols. The data repository 118 may store, but not limited to, data received from the processing unit 102, the communication means 1026, a healthcare professional, the user inter face 116, the user devices 1161, and the medical devices 106. It is also envisaged to store predetermined parameters. In one embodiment of the present invention, the processing unit 102 may include AI and deep learning-based trained models using the above data, to analyze and update the database based on the received data from the medical devices 106, processing unit 102 or the data received from the internet in real-time.

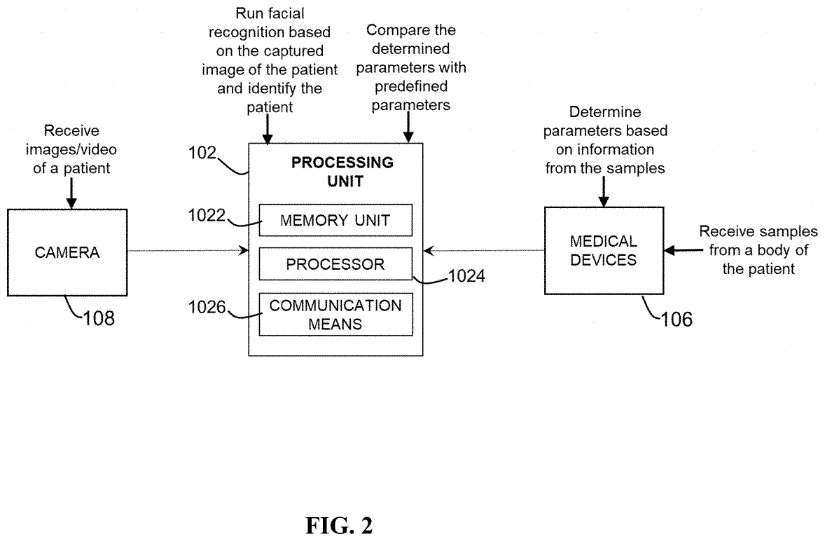

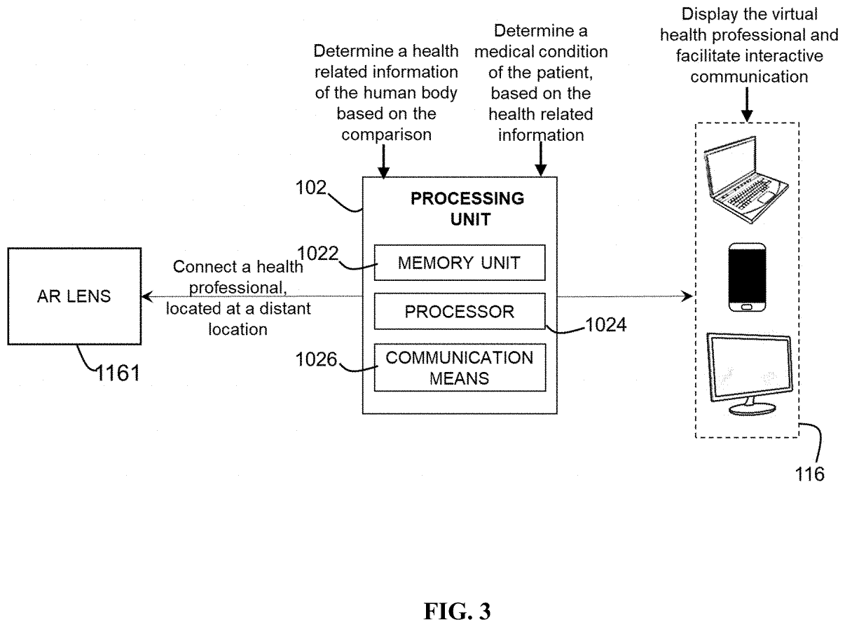

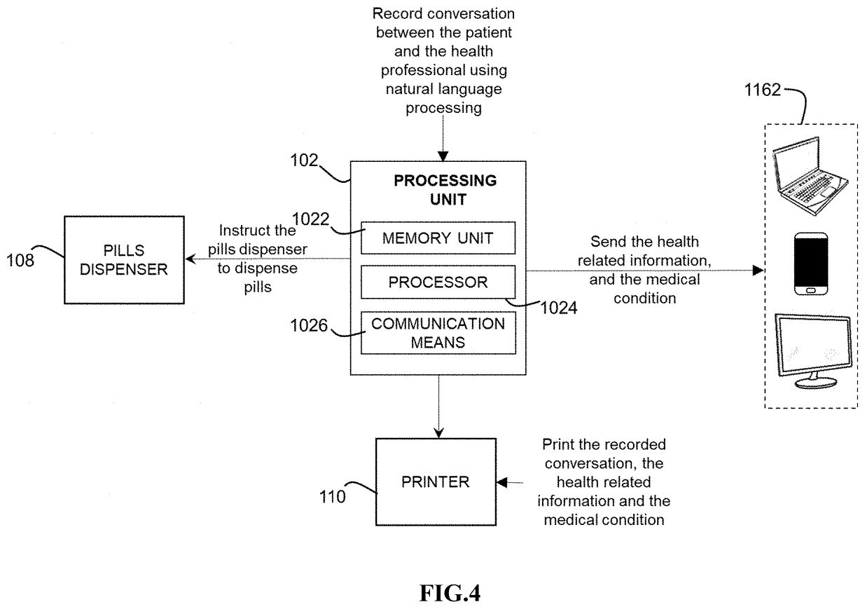

FIG. 2 illustrates information flow using system 100 of FIG. 1, in accordance with an embodiment of the present invention. The working of the present invention would be more easily understood with reference to the exemplary implementation shown in FIGS. 2, 3, and 4. As shown in FIG. 2, first, the camera 104 is configured to receive images/video of a patient. The processing unit 102 operably connected with the memory unit 1022, the processing unit 102 obtaining the machine-readable instructions from the memory unit 1022 and being configured by the machine-readable instructions to run facial recognition based on the captured image of the patient and identify the patient. In an additional or alternative embodiment, the processing unit 102 is further configured to determine emotions, and mental state of the patient based on the captured images/video using facial recognition.

The processing unit 102 may compare the captured image with predetermined images taking one or more markers on faces to determine the emotions, and mental state of the patient. For example, a patient living in a remote area having no access to hospitals or clinic may enter the portable healthcare system 100 installed at the remote location. The patient may enter the installation and get his/her image/video clicked/recorded by the camera 104 installed at entrance of the installation. The image is sent to the processing unit 102 for processing to run the facial recognition based on the captured image and identify the patient. The processing unit 102 may use biometrics to map facial features from the captured image/video. The processing unit 102 may compare information with a database of known faces to find a match. The facial recognition, in this way, may help verify the patient's identity the emotions, and the mental state.

Further, after identification of the patient, the emotions, and the mental state, as shown in FIG. 2, the patient is subjected to the point-of-care testing. The point-of-care testing has the one or more medical devices 106. Any medical device of the one or more medical devices 106 configured to receive one or more samples from the body of the patient. The one or more samples such as, but not limited to bodily fluids, tissue, mucus, DNA, urine, blood, saliva, sweat etc. The one or more samples are received by any of the medical devices 106. For example, the patient, after being identified at the portable healthcare system 100, may submit his/her samples at the point-of-care testing using the one or more medical devices 106 mounted at the installation. Let's say, the medical device is blood collection device installed at the portable installation configured to draw blood from the patient. The blood may be automatically drawn from the patient using the automated blood collection device.

Further, the medical devices 106 may determine one or more parameter based on information from the one or more samples and the body. The one or more parameters may be selected from a group comprising, but not limited to, body temperature, heart rate. SpO2, platelet count, salivary flow, pH and buffering capacity of saliva, Glucose, Bilirubin, Ketone, Specific Gravity, Blood. pH, Protein, Urobilinogen, Nitrite and Leukocyte from urine for testing of hematology, systolic blood pressure, blood gas, pathogens of certain infectious diseases, pregnancy, cardiac markers, and glucose. For example, the medical device such as the blood collection device may determine an amount of a platelet count as 130,000. Further, for example, the patient may be tested with the oxygen meter to determine a level of oxygen in the body of the patient as 89%.

As shown in FIG. 2, in accordance with an embodiment of the present invention, the processing unit 102 is configured to compare the determined one or more parameters with predefined parameters. The predetermined parameters may be a value or a range of values of the one or more parameters of an ideal healthy body. For example, the predetermined parameters such as the body temperature may be 96° F. to 98° F. Further, for example another predetermined parameter such as heart rate may be 67 beats per minute to 110 beats per minute. Furthermore, for example, the predefined parameter such as the platelet count may be in range of 150,000 to 450,000 platelets per microliter of blood.

After the comparison, as shown in FIG. 3, the processing unit 102 is configured to determine a health-related information of the human body based on the comparison. Continuing the example above, on detecting the oxygen level at 89%, the processing unit 102 may determine the health-related information of the human body as lack of oxygen. Further, for example, the processing unit 102 may determine the health-related information of the human body as lack of platelets based on the comparison. Similarly, for example, the heart rate monitor may detect heart rate of the patient as 140 beats per minute. After comparison, the processing unit 102 may determine the heart rate as unusually high.

After the determination of the health-related information of the human body, as shown in FIG. 3, the processing unit 102 is configured to determine a medical condition of the patient, based on the health-related information. For example, where the oxygen level of the patient is detected as low, the processing unit 102 may determine a corresponding medical condition of the patient as common cold or viral infection. Furthermore, where the processing unit 102 determine the health-related information of the human body as unusually high heart rate, the processing unit 102 may determine the medical condition of the patient as any of, but not limited to, fever, fear, stress, or anxiety related to the patient. Further, for example, because of low platelet count, the processing unit 102 may determine the medical condition as, but not limited to, immune thrombocytopenia. In accordance with an embodiment of the present invention, as shown in FIG. 4, the communication means configured to send the health-related information, and the medical condition to the one or more user devices 1162 held by the patient, and/or health organizations.

Next, returning to FIG. 3, the processing unit 102 is configured to connect a health professional, located at a distant location, through an augmented reality (AR) lens 1161 with the patient. The communication means 1026 is configured to establish communication network 114 by using satellite communication to ensure uninterrupted communication between the patient and the health professional. The installation may comprise antennas to boost signals to ease the satellite communication. For this purpose, the installation may comprise hardware for producing augmented reality such as holograms, head mounted device, or projectors to create an avatar of the health professional located at a distant location, in an augmented reality space. In accordance with an embodiment of the present invention, the user interface 116 is configured to display the virtual health professional and facilitate interactive communication between the virtual health professional and the patient to convey the health related information and the medical condition to the patient. The health professional may brief the patient about the health related information and the medical condition of the patient in the conversation with the patient.

Further, as shown in FIG. 4, the processing unit 102 is configured to record conversation between the patient and the health professional using natural language processing (NLP). The NLP may facilitate interpretation of the conversation between the health professional and the patient using speech recognition. The conversation may be recorded in audio format or may be converted into text. The conversation may contain the information regarding the medical condition of the patient and the medications for the medical condition determined. As shown in FIG. 4, in accordance with an embodiment of the present invention, the recorded conversation, the health-related information and the medical condition may be printed using the printer 110. The printed record may be auto generated after the conversation with the health care professional.

In accordance with an embodiment of the present invention, based on the determined medical condition and the health-related information, the processing unit 102 may suggest a prescription of medication. Further, based on the prescription, as shown in FIG. 4, the processing unit 102 may instruct the pills dispenser 108 to dispense the one or more pills. For example, the ECG analyzer detects blockage in the arteries, the processing unit 102 may suggest medication as blood thinners. In this case, the processing unit 102 may instruct the pills dispenser 108 to dispense the pills having blood thinners to treat the patient.

In accordance with an embodiment of the present invention, the conversation recorded between the patient and the health professional using natural language processing is used to create an automatic medical advisory for other patients having same medical issues. For example, any other patient, who visit such portable healthcare system 100, with similar health condition, based on the health-related information of that other patient, the processing unit 102 may suggest the medical advisory generated from previous diagnosis of the patient. Further, the processing unit 102 may instruct the pills dispenser 108 to dispense the prescribed medication based on the medical advisory. This way, in case of absence of any health care professional, the portable health care system 100 may diagnose a patient and give the medication.

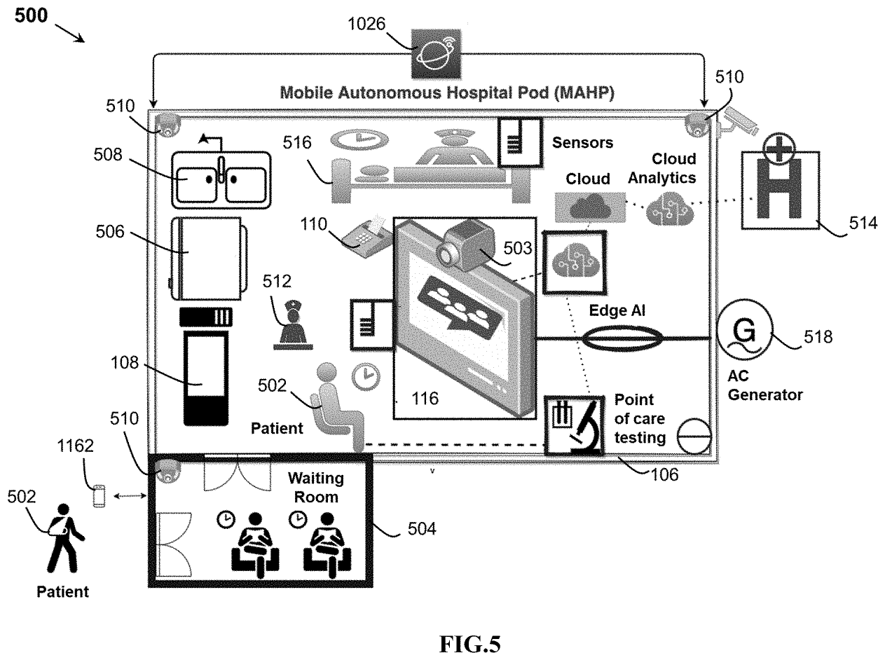

FIG. 5 illustrate an installation 600 implementing the system 100 of FIG. 1, in accordance with an embodiment of the present invention. As shown in FIG. 5, the patient 602 may be provided with a waiting area 604 for their turn at the installation 500. Further, as shown in the FIG. 5, the installation 600 may have facilities to accommodate sitting for the patient 502. The patient 502 may be subjected to point of care testing using the one or more medical devices 106 and sensors. Further, the installation 600 may be provided with a web cam 603 and the user interface 116 to have a conversation with the health care professional or hospital 614. The conversation may be established using the internet over the satellite communication 1026.

The healthcare professional or hospital 514 may receive the one or more parameters, the health care information and/or the medical condition of the patient 602 extracted by the one or more medical devices 106 and the processing unit 102 via may be, but not limited to, the satellite communication 1026. Further, as shown in FIG. 5, the installation 600 may be provided with a bed 516 and attended by a health attendant 612. For keeping the fluid and other perishable samples or medication, a refrigerator 506 may be provided. Further, as shown in FIG. 5, to rinse the patient 502, a sink 608 for cleaning and discarding the waste is provided. Further, as suggested by the processing unit 102 and/or the healthcare professional, the pills from the pill box/dispenser is dispensed to the patient 502. The printer 110 connected with the processing unit 102 may record and print the conversation using natural language processing. The installation 600 may be powered using the solar power generator. However, for constant generation of power, an AC generator may be used. Further, inside the installation 600, as shown in FIG. 5, one or more monitoring cameras 610 may be installed connected with the satellite communication 1026. The monitoring cameras 610 may provide live feed inside the waiting room and the installation 600 such as activities of the patient 502 and the medical attendant 512 via the satellite communication 1026.

It will be appreciated by a person skilled in the art that functioning of the invention may also be performed without following any strict order, and by one or more computer processor 1024 executing a program tangibly embodied on a computer-readable medium to perform functions of the invention by operating on input and generating output. Suitable processors include, by way of example, both general and special purpose microprocessors. Generally, the processor 1024 receives (reads) instructions and data from a memory unit 1022 (such as a read-only memory unit 1022 and/or a random-access memory unit 1022) and writes (stores) instructions and data to the memory unit 1022. Storage devices suitable for tangibly embodying computer program instructions and data include, for example, all forms of non-volatile memory unit 1022, such as semiconductor memory unit 1022 devices, including EPROM, EEPROM, and flash memory unit 1022 devices; magnetic disks such as internal hard disks and removable disks; magneto-optical disks; and CD-ROMs. Any of the foregoing may be supplemented by, or incorporated in, specially designed ASICs (application-specific integrated circuits) or FPGAs (Field-Programmable Gate Arrays).

A computer can generally also receive (read) programs and data from, and write (store) programs and data to, a non-transitory computer-readable storage medium such as an internal disk (not shown) or a removable disk.

One or more components of the invention may be described as modules/units for the understanding of the specification. For example, a unit/module may include self-contained component in a hardware circuit comprising of logical gate, semiconductor device, integrated circuits or any other discrete component. The unit/module may also be a part of any software program executed by any hardware entity for example processor. The implementation of module/unit as a software program may include a set of logical instructions to be executed by a processor or any other hardware entity.

Additional or less modules/units can be included without deviating from the novel art of this disclosure. In addition, each unit can include any number and combination of sub-units, and systems, implemented with any combination of hardware and/or software units.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and the appended claims.

1. A portable healthcare system installed at a remote location having limited internet connectivity, the system comprising:
a camera configured to receive images/video of a patient;
a point-of-care testing having one or more medical devices wherein any medical device of the one or more medical devices configured to:
receive one or more samples from a body of the patient;
determine one or more parameters based on information from the one or more samples.
a pills dispenser, configured to dispense one or more pills;
a memory unit configured to store machine-readable instructions; and
a processing unit operably connected with the memory unit, the processing unit obtaining the machine-readable instructions from the memory unit, and being configured by the machine-readable instructions to:
run facial recognition based on the captured image of the patient and identify the patient;
compare the determined one or more parameters with predefined parameters;
determine a health-related information of the human body based on the comparison; and
determine a medical condition of the patient, based on the health related information;
connect a health professional, located at a distant location, through a augmented reality (AR) lens with the patient;
record conversation between the patient and the health professional using natural language processing; and
instruct the pills dispenser to dispense the one or more pills.
a communication means configured to establish a communication network which is configured to send the health related information, and the medical condition to one or more user devices held by the patient, and/or health organizations.
a user interface, configured to display the virtual health professional and facilitate interactive communication between the virtual health professional and the patient to convey the health-related information and the medical condition to the patient;
a solar power generator configured to provide power;
a printer configured to print the recorded conversation, the health-related information and the medical condition;
wherein the augmented reality (AR) lens configured to project the health professional image as a hologram;
wherein the conversation recorded between the patient and the health professional using natural language processing is used to create an automatic medical advisory for other patients having same medical issues; and
wherein the communication means configured to establish communication network by using satellite communication to ensure uninterrupted communication between the patient and the health professional.

2. The system as claimed in claim 1, wherein the one or more medical devices are selected from a group comprising oxygen meter, electronic blood pressure (BP) monitor, thermometer, uric acid/UREA profiler, urine analyzer, glucose analyzer and cardiovascular analyzer, ultrasound scanner, an ECG analyzer, or a combination thereof.

3. The system as claimed in claim 1, wherein the one or more parameters are selected from a group comprising body temperature, heart rate, SpO2, platelet count, salivary flow, pH and buffering capacity of saliva, Glucose, Bilirubin, Ketone, Specific Gravity, Blood, pH, Protein, Urobilinogen, Nitrite and Leukocyte from urine for testing of hematology, systolic blood pressure, blood gas, pathogens of certain infectious diseases, pregnancy, cardiac markers, and glucose.

4. The system as claimed in claim 1, wherein the processing unit is further configured to determine emotions, and mental state of the patient based on the captured images/video using facial recognition.
